(19) United States
(12) Patent Application Publication
(10) Pub. No.: US 2021/0389266 A1
(43) Pub. Date: Dec. 16, 2021

(54) ELEMENTS AND COMPOUNDS MIXTURE DETECTION AND MEASURING SYSTEM

(71) Applicant: BTTRIGTICAL PTE LTD, Singapore (SG)

(72) Inventors: Meng Wye Yong, Singapore (SG); Xiaoling Wang, Singapore (SG)

(21) Appl. No.: 17/250,937

(22) PCT Filed: Sep. 25, 2019
(86) PCT No.: PCT/SG2019/050491

§ 371 Date: Mar. 31, 2021

(30) Foreign Application Priority Data
Sep. 28, 2018 (CN) CN 209086190 U

(51) Int. Cl. G01N 27/22; G01F 23/00; G01F 23/296

(52) U.S. Cl. CPC G01N 27/228; G01N 27/223; G01K 7/021; G01F 23/296; G01F 23/0076

(57) ABSTRACT

The invention relates to a mixture measuring system to detect and measure the composition of elements and compounds in a mixture. The system is connected to a cloud server, and it has a canister housing structure in which a sample taking cylinder is inserted to measure the capacitance of the mixture. The system comprises a sonar to measure the volume of the mixture in the cylinder, a gyroscope frame structure which supports a gyroscope stabilizer and other modules including a thermoelectric module, a heat dissipation device, a temperature sensor, a humidity sensor and data processing control. The system enables instant and accurate detection of the composition in the mixture.

### FIELD OF INVENTION

1. The invention is in the field of the measurement instrument and the invention is an element and compound mixture detection and measuring system that detect and measure the precise composition of all types of material, particles, elements, and compounds. It is able to accurately identify, detect and measure the composition of 2 or more of these elements and compound in the form of liquid(s), solid(s), powder(s), dissolved gas(es) or a mixture of two or more group of the mixtures, For example, the equipment is capable of measuring the exact water content of crude or processed upstream oil. It can also be used to measure the different types of water (oxygenated, mineral, hydrogenated, alkaline, etc, etc).

It is known that the accurate determination of the level of liquid in a measuring cup is an important input for an accurate calculation of an element or multiple elements in a liquid mixture or mixtures. The exact composition of each or more element in the mixture is a derivation of many calculating factors, including the sample mass, volume, and concentration. The invention has an extensive application need in various industries, including the oil and gas industry that demand extremely high accuracy of oil and multiphase fluid sample measurement. These measurements are needed in measuring the water content in oil and multiple mixtures of liquid to gas, or elements in liquid such as water in oil, taken from the oil well in the upstream processing from onshore or offshore oil wells or downstream process in refineries, petrol stations, etc. A gyroscopic stabilizer also allows measurement done in floating vessels, ships, floating platforms of uneven terrain when any slope of uneven platforms will affect the precision reading.

The invention is also suitable for laboratory measurements of liquid, whether in emulsion, slurry and chemical solution from various industrial processes, such as food and beverage production, petrol-chemical, pharmaceutical, drug production, and chemical laboratory. In many industries, the precise and exact volume and composition are extremely important in the production processes. In the preparation of emulsification solution, an exact concentration of impurities in liquids must be detected and measured.

### BACKGROUND OF INVENTION

1. To precisely detect and measure the composition of 2 or more liquids in mixture is a momentous challenge. Many methods like Karl Fischer's that reagents or distillation technique are used to determine the water content of crude oil or processed oil. The invention uses a purpose-built canister system to measure the capacitance of sample mixture (liquid, solid, powder, dissolved gases, chemicals). The canister system detects the electrical signature or capacitance of each component in mixture through the use of electrical conductivity. The canister system is made up of a housing structure that hosts the cylinders, with conductive plates. Depending on the type, volume of the component in the mixture, by adjusting the physical structure of the cylinder and the component that make up the cylinder, it is able to measure the mixture in finding out its exact composition.

Measurements of fluid sample in a mixture rely on some fundamental inputs, like level, volume, and its precise measurement are important to calculate the mass, density, concentration, composition of the samples. Accurate determination of fluid sample volume and composition is a challenge due to many reasons. The most common reason being that capacitance determination of each component in a mixture is extremely sensitive to changes in electrical and environmental pollutants and interference. The other reason being the sensitivity of the device to detect the capacitance or cycle of electrical changes can be difficult that was caused by the material used to construct the detection devices.

1. Volumetric cylinders and flasks are often used to measure the volume of liquids by reading the gradation line printed on the containers. These reading, based on printed line, is only approximation of the actual volume and it cannot measure to micro-meter. Hence, the accuracy of the volume reading limits the precision of the final measurement of the mixture.

Calibrated float and level sensor are also used to find the liquid level when the platform that holds the system or devices are uneven. As container must be placed on even surface platform, a tiled container will result in wrong measurement. When the container is tilted with an angle, liquid sloped to the direction of tilt. For this reason, the conventional approach cannot be used on floating vessels like ships or floating vessels, or in locations with complex terrains like hills or mountains.

In today capacitive technology, the mixture is measured by using probing capacitive sensors. This method is highly inaccurate with capacitive sensors contacting the samples. In mixture every component in the mixture can interfere with each other. When components are in contact, each contact conducts electricity that is erratic, causing fluctuation to the capacitance reading. This method has been known to produce extremely erratic and inaccurate results due to the polarization phenomenon. This phenomenon is caused by the contact between the electrodes or conductive plates and the samples. There are other disadvantages with today technology, many of the devices are highly immobile and not portable due to its size. Devices cannot be deployed in remote or capable to be used on-site if it is not portable.

The time to measurement using devices with today technology is extremely long, this is caused by the use of reagent and also pe-sample preparation processes. These instruments are very expensive and have high error rate or range of range deviation btw 1-15%. If the mixture contains conductive and non-conductive material, it is extremely incapable to identify, cannot measure the composition of the mixture elements accurately. In some tests, contact with compounds is harmful, the operating environment is highly toxic, especially if the operators are to combine various mixture of chemical that is safety hazards.

### DETAILS OF THE INVENTION

The key details of the invention are described as follows;

### 1. Purpose, Usages, and Structure of the System

1. A mixture is a combination of liquid, solid, powder, minerals or dissolved gases. The invention combined various modular sub-systems to detect, measure the composition of the elements, compounds in the mixture. The measurement is performed by placing the mixture in a cylinder. The cylinder is inserted into a canister housing in the sample taking canister system. A gyroscope with an attached sonar is position at the opening of the canister structure. The sonar is to measure the volume of the mixture inside the cylinder. The gyroscope and the sonar could overcome the uneven platform that holds the measuring system causing the system to be tilted. The gyroscope and the sonar system helps to ensure that the accuracy of the volume reading is maintained. Conventionally, the bull's eye spirit level is used to level the instrument in two dimensions. When the gas bubble is centered precisely inside the markers, the instrument was checked for its horizontal position.

### 1. The Sample Taking Cylinder

1. has an insulating layer isolating the mixture from the conductive plate. As the volume of the mixture is measured through the sonar measurement system, the canister structure is stabilized and leveled by the gyroscope stabilizer. During which the sonar makes detection of the volume level in the cylinder. By combining the volume, stabilized by the gyroscope, the exact composition of the mixture can be accurately measured.

### 1. Modular Structure.

1. The measuring system has the following technical features in the detection and measurement process. The device has an internet connection to a cloud server. The measuring system is developed on modular form and comprises many interconnected modules. The canister module which has a sample taking the cylinder is to measure the capacitance of the sample mixture. A sonar measuring module is to measure any liquid level and/or volume of the mixture in the cylinder. A thermoelectric module is to maintain the sample mixture at a constant temperature and radiator, temperature sensor, humidity sensor, data processing control board and power supply are connected to these modules.

There is a gyroscope frame at the top of the canister housing. A gyroscope stabilizer is placed on the gyroscope frame and place on top of the housing where the canister structure is located.

### 1. Detection and Measuring Methodology.

1. At the housing structure where the gyroscope and the canister structure is located. A sample taking cylinder can be inserted into the canister housing. The sample taking cylinder contains one or more conductive plates, insulating layers, and a PCB circuit board. The conductive plate and an insulating layer placed in the cylinder are located between the inner sidewall of the cylinder body and the sample mixture. The conductive plate can make up of many plates in one or more groups, it is connected to the PCB circuit board. During measurement, electricity is passed to the conductive plate, which is electrically charged. As the electrical field is generated between conductive plates, the PCB board measures the charging and discharging cycle of these conductive plates, which is the sum of charging time and generating time, so as to find the capacitance of the mixture in the cylinder. PCB board calculate and record the capacitance and pass the data to the cloud server. It will be compared to the standard capacitance information chart stored in the cloud server and the data is compared to find the exact composition of the mixture. The insulation of the sample mixture from the conductive plate, ensure that the mixture makes no contact with the conductive plate, effectively reduces the interference, significantly improving the measurement of the sample mixture.

### 1. Central Processing Unit

1. The PCB board has a capacitor pole interface, noise processing module, capacitor digital conversion module, and microcontroller I. The conductive plate is connected with the microcontroller I through capacitor pole interface, a noise processing module, capacitor digital conversion module in measuring the capacitance cycle. The output of microcontroller I am connected to data processing control board.

### 1. The Sonar Measurement System

1. The system made up of ultrasonic sensor, driving module, signal processing module, ADC conversion module, microcontroller II and DAC conversion module; ultrasonic transmitter of ultrasonic sensor is connected with microcontroller II through driving module and ADC conversion module in turn; ultrasonic receiver of ultrasonic sensor is connected with microcontroller II through signal processing module. The output port of the microcontroller II is connected with the DAC conversion module.

### 1. The Data Processing Control Board and Temperature Moderation Systems

1. The system has central processing module and a communication module; the input end of the central processing module is connected to the sonar measurement system, temperature sensor, and humidity sensor respectively; the output end of the central processing module is connected to the thermoelectric module and the heat dissipation device respectively; the output end of the central processing module is also connected to the cloud server through the communication module, and the communication module is connected to the cloud server through the communication module. RS-485 serial communication is adopted.

The output terminal of the central processing module is connected to a display.

### 1. The Levelling Deign and Structure.

1. The gyroscope stabilizer is connected to the gyroscope frame structure through bearings. The set-up of the gyroscope stabilizer keeps the measuring system and the entire device on even level and stable. The gyroscope system solves the problem of major measurement error when the measuring system was placed on uneven surface. eg when measuring the volume of liquid or powder samples on ships or floating boats, hills or mountainous areas etc. Sonar measurement system is calibrated and placed under the cover to the housing to detect the volume after compensation and correction by the gyroscope. With these design, accuracy can reach 3 microns and 50 ppm. The gyroscope and sonar detection systems are widely used for volumetric measurement of various liquids, solids, powders, and dissolved gases.

### 1. Temperature Equaliser.

1. There is a ventilation fan and heat sink. The thermoelectric module uses a semiconductor heater and cooler, which keeps the sample in the cylinder at a constant temperature (e.g. 20 C), thus preventing any changes in temperature affecting the capacitance reading of the sample.

On prior art comparison, the advantages and invention of this measuring system are:

(1) The measuring system is to detect mixtures of elements and compounds, in different type and forms, including liquid, gas, particles, powders, and elements. The invention has a canister housing and consist of a cylinder with a conductive plate and an insulating layer in the sample taking cylinder. The sample mixture is isolated from the conductive plate in the cylinder. The conductive plate does not interfere with the measured sample in the course of use, and when measuring the capacitance of two or more mixture samples, the influence of conductivity on the measuring results can be effectively reduced; moreover, the insulating layer is hydrophobic and oil-draining.

(2) The measuring System has a calibrated ultrasonic sensor and a gyroscope. A gyroscope stabilizer, with high precision, can measure liquid that has high transmittance or has high flammability. The inclusion of the gyroscope stabilizer solves the problem of large measurement error caused by poor placement of a volume measuring device or taking measurements in uneven or unstable environment, such as measurement in a moving vehicle and ship.

(3) The measuring system can measure two or more mixtures quickly and accurately. The precise composition of the compound can reach 0.1% accuracy, and the measuring time is as short as 1-15 minutes. Moreover, there is no need to add chemical reagents or additives, hence saving cost and also preventing error caused by unstable of inaccurate use of reagents.

### DRAWINGS DESCRIPTIONS

For a detailed description of the embodiments of the invention, reference will now be made to the accompanying drawings in which

FIG. 1 is a schematic diagram of the measuring system.

FIG. 2 is the schematic modular diagram of the measuring system.

FIG. 3 shows the structure of the sample taking cylinder.

FIG. 4 is the schematic diagram of PCB circuit board.

FIG. 5 is a schematic diagram of the connection between the sonar measurement system and the sample taking cylinder.

FIG. 6 is the schematic diagram of the sonar measurement system.

FIG. 7 is the circuit diagram of the data processing control board.

FIG. 8 is the circuit diagram of the sonar measurement system.

FIG. 9 is a circuit diagram of a temperature sensor or a humidity sensor.

FIG. 10 is the circuit diagram of the thermoelectric module controlled by the central processing module.

FIG. 11 is a circuit diagram of the central processing module controlling the ventilation fan.

### EMBODIMENTS, EXPLANATION OF DRAWING AND DIAGRAM AND PRACTICAL USES

1. The following are details explantion and description to the illustrations, drawings and provide better clarification to these drawings. These embodiments intend to clarify the intended uses of the measuring system but not limiting to that were not mentioned.

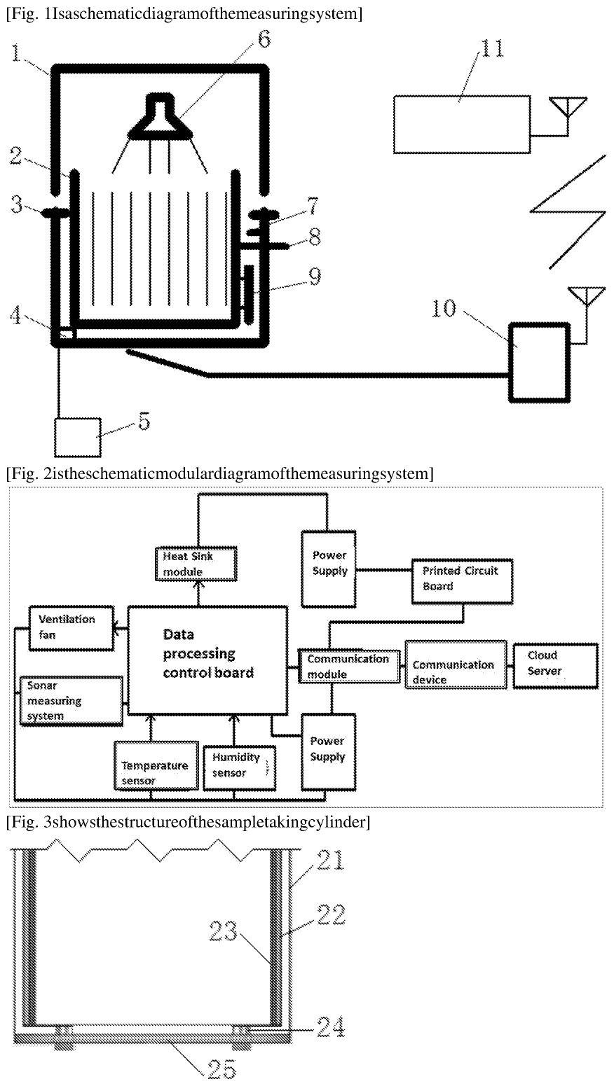

In FIG. 1 or 2, which is a schematic diagram and a modular schematic diagram of the measuring system. The diagrams depict a wireless communication module (10) connected to a cloud server (11). The measuring system has a canister housing structure that house the sample taking cylinder (2). The purpose of these modules is to detect the capacitance of the mixture in the cylinder (2). A sonar measuring system (6) is included in the main measuring system to detect and measure the liquid level and determine the volume of the mixture in the sample taking cylinder (2). A thermoelectric module (9), a heat sink (4), a temperature sensor (8), a humidity sensor (7), a data processing control board and the power supply (5) are connected to the sample taking cylinder (2). The sonar measurement system (6), the thermoelectric module (9), the heat sink (4), the temperature sensor (8) and the humidity sensor (7) are linked to the data processing control board and the power supply (5). The canister housing structure has a gyroscope rack structure (3), a gyroscope stabilizer (63) and a cover (1). The measuring instrument is sealed with a cover (1) during the measuring process.

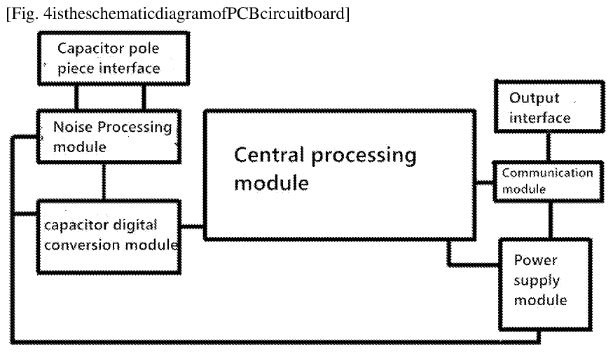

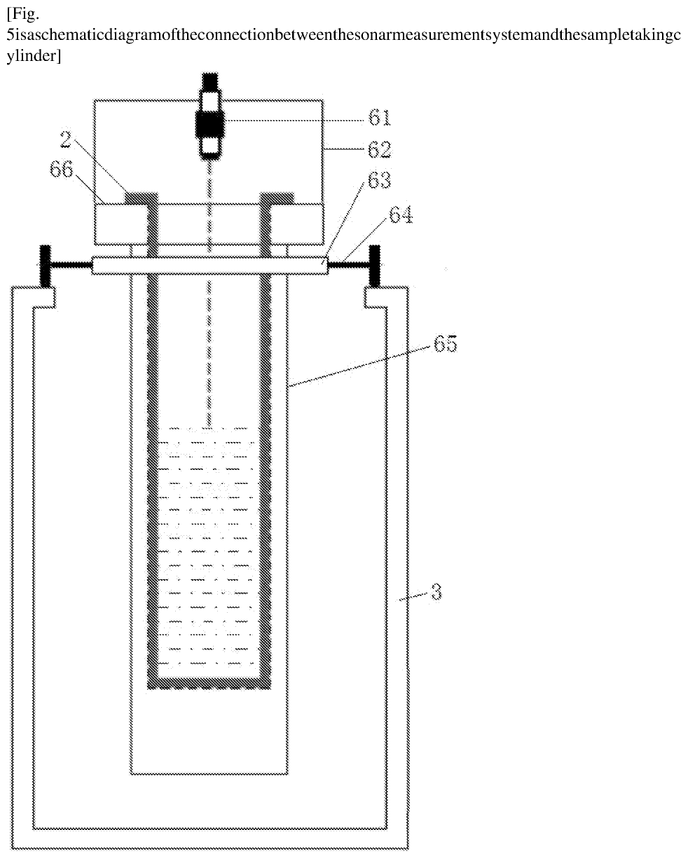

In FIG. 3 or FIG. 4, shows a sample taking cylinder (2), a canister housing (21) and conductive plate (22). There is an insulation layer (23) and a PCB board (25). The Conductive plate (22) and insulation layer (23) are located in the cylinder (21) and curved around the inner wall of the cylinder (21). The conductive plate (22) is connected through a connecting plate (24) to the PCB board (25) and both are placed at the outside bottom of the sample taking cylinder. The PCB board (25) has a capacitor chip interface, noise processing module, capacitor digital conversion module and microcontroller I. Conductive board (22) is connected with microcontroller I through capacitor chip interface, noise processing module and capacitor digital conversion module to measure capacitance cycle. The output terminal of microcontroller 1 is connected to the data processing control board. A standard set of capacitance data formulated from measuring a range and variation of compounds, elements, and liquid mixtures are stored in the cloud server. The capacitance recorded from the measuring system is compared against the on-line standard data. The capacitance is measured as electricity conducted through the conductive plate (22) that generate electrical fields. The PCB circuit board (25) determine the charge and discharge cycle of the conductive plate (22), the charging time and generating time, and to find the capacitance of each component in the mixture. Data processed in PCB circuit board (25) is passed through the communication and capacitance recorded from the sample taking canister (2) module is compared to the standards stored in cloud server (11). The data is processed using algorithms to find the precise measurement of the composition of the mixture. The insulating layer (23) design is crucial in the effective reduction of conductivity interference and input to the measurement results. Depending on the material that make-up of the insulation layer (23) and thickness, between 10%-95% of capacitance different are usually noticed comparing to traditional method of measurement. The improved sensitivity of the measuring system is extremely important if needed to measure composition with only less than 50 ppm accuracy tolerance. The Insulation layer (23) is made of inert material, such as PTFE or PI film, which is hydrophobic and oil-thinning. Its thickness is 10-1000 um. By adjusting the thickness of the insulation layer (23) relative to type of mixtures and the components to be measured, it can be designed to measure different level of accuracy. The customization and design of the insulation layer (23) will determine how sensitive the measuring system can be constructed and how precise the measurements are needed.

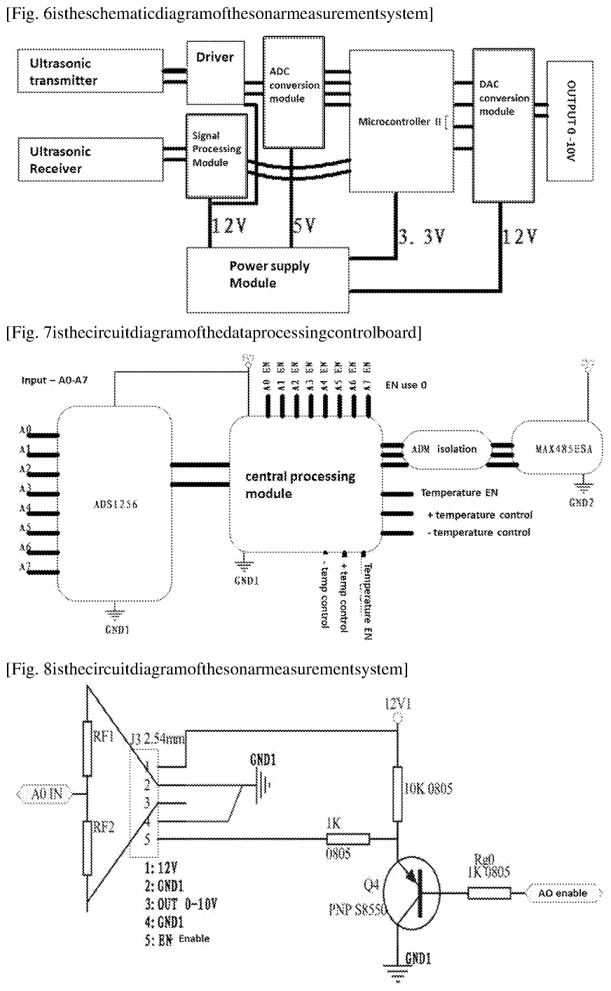

In FIG. 5 and FIG. 6, an embodiment of a sonar measurement system (6) is shown. The sonar measurement system has an ultrasonic sensor (61), driving module, a signal processing module, ADC conversion module, microcontroller II and DAC conversion module, The ultrasonic transmitter is connected with microcontroller II through driving module and ADC conversion module. The ultrasonic receiver of ultrasonic sensor (61) is connected with microcontroller II through a signal processing module. The output port of the device is connected with the DAC conversion module. Sonar measurement system 6 is calibrated and has a sealed cover. Its accuracy can reach 3 microns meter. It is widely used for volumetric measurement of various liquids, solids, powders, and dissolved gases.

The structure schematic diagram sonar measuring system (6) and sample taking cylinder (2) is shown in FIG. 5. The sonar detection housing has a gyroscope bracket (3), a gyroscope stabilizer (63) is installed on the gyroscope bracket (3), and a gyroscope stabilizer is connected to the gyroscope bracket (3) using a bearing structure (64). The Gyroscope structure that has a gyroscope stabilizer (63) which is placed onto the gyroscope frame (3) through bearing (64) is to ensure the measuring system when placed on an uneven platform, has its canister structure kept at a leveled level and stable position. It solves the problems when the measuring system is placed on uneven ground, eg on a ship or a floating platform, slope, etc

A Sonar Sensor bracket (62), canister structure (65) house the sample taking cylinder 2 that holds the sample. The sample taking cylinder (2) is inserted into sensor bracket (62) and slipped into the canister structure. The outer edge of the cylinder (2) is locked on the support structure (66) and sensor bracket (62). It also includes an open or closed sealing cover. One side of the sealing cover is connected at the top of sensor bracket (62). The underside of the sealed cover is attached with an ultrasonic sensor (61), that measures the distance between the sample mixture in the sample taking cylinder (2) and the ultrasonic sensor (61) to find the volume of the sample mixture. The sealed cover structure protects the sample mixture from contamination when closed. It also protects the operator when toxic and harmful liquid is measured.

The sample mixtures are placed into the sample taking cylinder (2). The cylinder (2) that has conductive plates and insulating layers that are specially designed for different types of components, mixtures, elements, and compounds measurements. The composition of the mixture is determined through processing the capacitance data taking into accounts of the volume, weights and concentration of the mixture detected in the sample taking cylinder (2). The volume of the mixture is measured by the sonar measuring system (6). The weight of the mixture measured determined by the sample taking cylinder. Taking the data from the volume and weight, the concentration of the mixture can be accurately determined. The measuring system can accurately determine the composition or combination of two or more liquids, solids, powders or dissolved gas ES. For example, it can measure the water content of crude oil or processed oils, and also can be used to measure the types of water, such as oxygen, minerals, hydrogenation, alkalinity, etc. The measuring system is widely applicable to the laboratory measurement of liquid, emulsion, slurry or chemical solution from various industrial processes such as food and beverage production, in petrochemical industry, in pharmaceutical production. In pharmaceutical production and chemical laboratories, it is crucial that the exact exact composition of the mixture is known in emulsion formula preparation or in solution. This enable the concentration or impurity content of the liquid to be identified

As shown in FIG. 7, the data processing control board has a central processing module and communication module. The communication module includes an isolation interface and MAX485ESA chip. The isolation module uses AMD2486 isolation chip in connecting and filter RS-485 communication. The central processing module uses STM32F series microcontroller that has input interfaces with the sonar measurement module (6), temperature sensor (8) and humidity sensor (7).

In FIG. 8, The circuit diagram of sonar measurement system (6) and central processing module are shown.

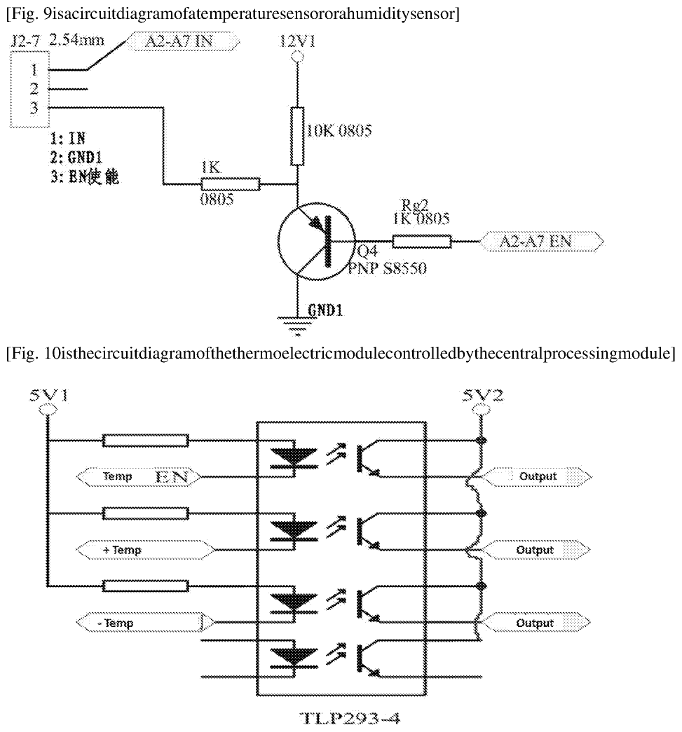

In FIG. 9, the circuit diagram of the temperature sensor (8) or humidity sensor (7) and the central processing module are shown. The output terminal of the central processing module is connected with the thermoelectric module (9) through the photoelectric coupler TLP293-4.

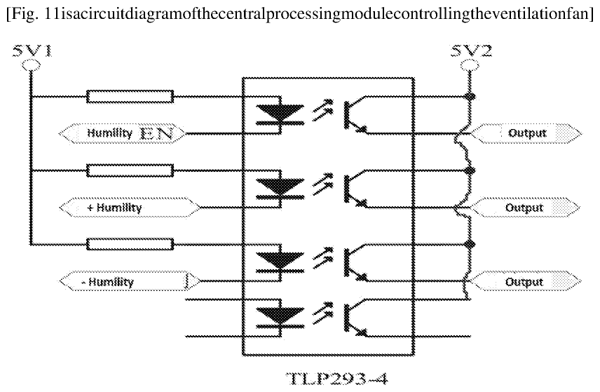

FIG. 10 is the circuit diagram of the thermoelectric module controlled by the central processing module. The thermoelectric module (9) uses a semiconductor heater to prevent changes in temperature to affect the capacitance reading of the components in the sample mixture. The temperature sensor (8) processes the temperature signal from the sample taking cylinder (2) and passed on to the central processing module. The central processing module controls the thermoelectric module (9) on receiving the temperature signal from sample taking cylinder (2). The standard constant temperature is 20 C and is adjusted to avoid temperature changes affecting the mixture capacitance reading from the sample taking cylinder (2). The ventilation fan is used in the heat dispersing device (4) to ventilate the measuring system. The output terminal of the central processing module is connected with the fan through the photoelectric coupler TLP293-4.

In FIG. 11, the humidity sensor (7) measures the humidity in the measuring system and transmit it to the central processing module. The ventilation fans open or closes according to humidity level, and the ventilation fans are to avoids damaging the equipment from overheating and affect the accuracy of the capacitance reading of the samples. The output terminal of central processing module is also connected with cloud server 11 through communication module and the output terminal of the central processing module is also connected with a display.

The measuring system measured the capacitance of elements and compounds in mixtures. The elements and compounds can be in liquid, solid, particular, gas or molecular forms.

1. The invention is a mixture measuring system to detect and measure the composition of elements and compounds in a mixture or mixtures. The mixture or mixtures can be in the form of compound, solid, powder, element and gas. It can also measure molecule and microorganism. The mixture detection and measuring system comprise multiple modules and structures that are interconnected with each other. The followings are features and systems that make-up of the measuring system:
a. The mixture measuring system has a cloud connection to the cloud server (11);
b. It has a structure the shape of a canister, (known as canister housing structure) in which a cylinder (2) can be inserted to measure the capacitance of the mixture
c. A sonar measurement module (6) for measuring the liquid level and/or volume of the mixture in a cylinder (2)
d. A thermoelectric module (9) for maintaining a constant temperature of the sample in the cylinder (2),
e. A temperature sensor (8), a humidity sensor (7), a data processing control board and a power supply (5).
f. The data processing control board and the power supply (5) are respectively connected to all modules including the cylinder (2), the sonar measurement module (6), the thermoelectric module (9), a radiator (4), the temperature sensor (8), the humidity sensor (7)
g. The gyroscope frame structure (3) is to support the gyroscope stabilizer (63).

2. The mixture detection and measuring system, in addition to that described in claim 1, has a canister structure that houses the sample taking cylinder (21). The sample taking cylinder (21) has conductive plate or plates (22), an insulating layer (22) and PCB circuit board (25) designed along the inner wall of the cylinder casing.

3. The insulation layer (23) which is located inside the cylinder has a thickness of between 10 to 1000 um.

4. The sample taking cylinder (21) has an insulating layer (22) the shape of an arc and is located at the inner side of the cylinder. The insulating layer and the shape of the conductive plate (6) are arc around the inside wall of the cylinder. The conductive plate is make-up of one or more plates in one or more group and is located between the insulating layer and the inner wall of the canister structure. The insulating layer is the outer most layer inside the cylinder and is next to the conductive plate that insulates the sample mixture from contacting the conductive plate

5. The mixture measuring system has a capacitor chip interface, a noise processing module, a capacitor digital conversion module, and a microcontroller one on the PCB circuit board (25). The conductive plate (22) is connected to the microcontroller one through a connecting plate and that the capacitor chip interface, a noise processing module and a capacitor digital conversion module are interconnected to detect the capacitance cycle of the measured compound or element in the sample mixture. The output terminal of the microcontroller one is connected to the data processing control board.

6. The mixture measuring system has a sonar measurement system (6). The sonar measurement systems have an ultrasonic sensor (61), driving module, signal processing module, ADC conversion module, microcontroller II and DAC conversion module. The ultrasonic transmitter of ultrasonic sensor (61) is connected with microcontroller II through the driving module and ADC conversion module in that ultrasonic signal sensing is detected. An ultrasonic receiver of the device (61) is connected with the microcontroller II through the signal processing module, and the output port of the microcontroller II is connected with the DAC conversion module.

7. In addition to all the claims described in claim 1 to claim 5, the mixture detection and measuring system has a data processing control board, an in-built central processing module, and a communication module; the input end of the central processing module is connected to the sonar measurement system (6), the temperature sensor (8) and the humidity sensor (7); and the output end of the central processing module is connected with the thermoelectric module (9) and the heat dissipation device (4); The output end of the module is also connected to the cloud server (11) through the communication module.

8. The output interface of the central processing module is linked to a screen display.

9. A gyroscope system that makeup of a stabilizer (63) that is mounted onto the gyroscope frame (3) and is connected to a bearing structure (64).

10. The mixture measuring system has a heat dissipation device (4) and a ventilation fan.

11. The mixture detection and measuring system have a thermoelectric module (9) and a semiconductor type heater and cooling device.
